(19) United States
(12) Patent Application Publication
(10) Pub. No.: US 2023/0321659 A1
(43) Pub. Date: Oct. 12, 2023

(54) MICROFLUIDIC CHIP FOR HIGH-THROUGHPUT CELL PAIRING AND FUSION

(71) Applicants: Massachusetts Institute of Technology, Cambridge, MA (US); Tecnologico de Monterrey, Monterrey (MX)

(72) Inventors: Mohammadmahdi Aeinehvand, Cambridge, MA (US); Sergio Omar Martinez-Chapa, Monterrey (MX); Joel Voldman, Belmont, MA (US)

(21) Appl. No.: 18/166,767

(22) Filed: Feb. 9, 2023

Related U.S. Application Data
(60) Provisional application No. 63/329,797, filed on Apr. 11, 2022.

(51) Int. Cl. B01L 3/00; C12M 3/06

(52) U.S. Cl. CPC B01L 3/502761; B01L 3/502715; C12M 23/16; B01L 3/502707; B01L 2200/0647; B01L 2300/0816; B01L 2400/0487; B01L 2300/0864; B01L 2200/027; B01L 3/50273

(57) ABSTRACT

Microfluidic chips, systems, and methods of making and using thereof are described. The microfluid chip includes a disc-shaped transparent layer containing a plurality of cell traps, pillars, and filers, and a support layer attached thereto. The microfluid chip has at least one inlet port in center of the transparent layer for receiving a sample of liquid and cells, and optionally a plastic cover. The microfluid chip can be designed to be suitable for the forces used for cell pairing/fusion in stationary and spinning format, or suitable for a particular cell fusion method such chemical and electrical methods. The microfluid chip is particularly suited for fusing dendritic cells and tumor cells for immunotherapy, or for generating hybridoma.

### CROSS-REFERENCE TO RELATED APPLICATIONS

This application claims benefit of U.S. Provisional Application No. 63/329,797 filed Apr. 11, 2022.

### STATEMENT REGARDING FEDERALLY SPONSORED RESEARCH

This invention was made with government support under CA259840 awarded by National Institutes of Health. The government has certain rights in the invention.

### FIELD OF THE INVENTION

The invention is generally in the field of microfluidic devices for cell fusion, methods of use, and the products thereof.

### BACKGROUND OF THE INVENTION

Cellular fusion provides a unique tool to combine genetic and epigenetic information of two cells. Since its first application in the 1960s, it has been mainly used to identify trans-acting factors that affect gene expression as well as to generate antibody-producing hybridomas (Blau H M, et al., Science. 230:758 (1985); Kohler G, et al., Nature. 256:495 (1975); Miller R A, Ruddle F H. Cell. 9:45 (1976)). More recently, the fusion of enucleated oocytes and embryonic germ cells (EGC) with somatic cells has provided definitive evidence for epigenetic reprogramming mediated via trans-acting factors (Tada M et al., Embo J. 16:6510 (1997); Wilmut I, et al., Nature. 385:810 (1997)). Besides EG cells, embryonic stem cells (ESC), and embryonic carcinoma cells (ECC) also have been proven to reprogram somatic cells (Matveeva N M, et al., Molecular Reproduction and Development. 50:128 (1998); Tada M, et al., Current Biology. 11:1553 (2001); Flasza M, et al., Cloning Stem Cells. 5:339 (2003)). In all these cases, fusion is the crucial step, but technical limitations in how fusion is carried out have prevented detailed studies of fusion-mediated reprogramming. As a result, the mechanisms by which the transcriptional program of a cell is altered after fusion, leading to nuclear reprogramming, remains largely unknown.

Fusion of cells can be induced biologically (viruses, receptors), chemically (Polyethylene Glycol), or physically (electric pulse), with the latter two representing the two most commonly used techniques (Kohler G, et al., Nature. 256:495 (1975); Miller R A, Ruddle F H. Cell. 9:45 (1976); Jahn R, Lang T, Sudhof T C. Cell. 112:519 (2003); Pontecorvo G. Somat Cell Genet. 1:397 (1975); Davidson R L, Gerald P S. Somat Cell Genet. 2:165 (1976); Vienken J, Zimmermann U Febs Lett.; 137:11 (1982); Zimmermann U, Vienken J. J Membrane Biol. 67:165 (1982)). Both chemical and electrical fusion rely on random cell-cell pairing and result in low overall fusion efficiencies, requiring antibiotic selection and lengthy subculturing to isolate the desired hybrids. Alternatively, single cells can be manually immobilized and then paired, resulting in precise fusion partners, but low numbers of fused cells (Stromberg A, et al., Proc Natl Acad Sci USA. 97:7 (2000)).

Improving the process of cell fusion lies in both the mechanism of initiating membrane fusion as well as in controlling how the cells are brought into contact and properly paired. There have been previous attempts using microfluidics for cell pairing, utilizing either flow-through or immobilization techniques to improve cell contact. Flow-through approaches, in which cells are brought into contact through AC fields or biotin-streptavidin coatings, demonstrate that higher membrane fusion efficiencies can be achieved (Tresset G, Iliescu C. Applied Physics Letters. 90:173901 (2007); Tresset G, Takeuchi S. Biomedical Microdevices. 6:213 (2004); Schut T C B, et al., Biophysical Journal. 65:568 (1993); Wang J, Lu C. Applied Physics Letters. 89:234102 (2006)). However, these approaches lack the ability to properly pair and fuse unmodified cells, and the overall yield of desired fusions remains low.

Immobilization techniques using hydrodynamic weirs or suction have demonstrated the ability to properly pair cells, and these devices have been used successfully for electroporation (Khine M, et al., Lab on a Chip. 5:38 (2005); Lee P J, et al., Applied Physics Letters. 86 (2005); Khine M, et al., Lab on a Chip. 7:457 (2007); Valero A, et al., Lab on a Chip. 8:62 (2007); Di Carlo D, et al., Anal Chem. 78:4925 (2006)), but thus far are incapable of pairing and fusing cells.

Microfluidic isolation and trapping of single-cells and cell clusters shows great potential in advancing medical and biological research (Weaver W M. et al., Curr. Opin. Biotechnol. 25, 114-123 (2014); Konry T et al., Annu. Rev. Biomed. Eng. 18, 259-284 (2016); and Charnley M. et al., Integr. Biol. 1, 625 (2009)). This is in part due to enabling investigations in cell-to-cell interactions and signaling, studies of cell heterogeneity in tumors, development of immunotherapies, investigations of drug responses, and regulation of stem cell differentiation (Luan Q et al., Biomicrofluidics. 2020 May; 14(3): 031502). In conventional cell studies, cells are studied in larger populations in which measurements can only reflect average values summed over multiple cell responses (Johann R M., Anal. Bioanal. Chem. 385, 408-412 (2006)). This approach, however, can lead to misinterpretations since it obscures important information about the cells and their statistical nature. In addition, cell growth is subject to multiple spatial and temporal cues including cytokine and protein gradients and cell-matrix or cell-cell interactions (El-Ali J. et al., Nature 442, 403-411 (2006)); therefore, better control over the cell environment is desirable when conducting in vitro experiments to achieve long cell viability.

Microfluidic trapping devices immobilize single cells and cell aggregates in a controllable and independent environment, which offers a way to conduct analysis at a single-cell level. These microfluidic trapping devices allow integration of cell culture with analytical devices (Dittrich P. S. and Manz A., Nat. Rev. Drug Discov. 5, 210-218 (2006)) as well as the ability to probe biochemical processes that govern cell behavior (Young E. W. K. and Beebe D. J., Chem. Soc. Rev. 39, 1036-1048 (2010)), which permits genetic (Zeng Y., et al., Anal. Chem. 82, 3183-3190 (2010); Easley C. J. et al., “Proc. Natl. Acad. Sci. U.S.A. 103, 19272-19277 (2006)), physiological (Chen M. B., et al., Lab Chip 13, 2591-2598 (2013); Ortega-Prieto A. M. et al., Nat. Commun. 9, 1-15 (2018)), and biochemical cell studies (Choi J. W. et al., Lab Chip 2, 27-30 (2002); Bilitewski U., et al., Anal. Bioanal. Chem. 377, 556-569 (2003)) on a single-cell to organ scale. Microfluidic trapping devices also offer a potential for 3D cell culture to better mimic the in vivo microenvironment, allowing researchers to study physical effects on cell function and behavior (Sackmann E K., et al., Nature 507, 181-189 (2014)) and with the scale-down of research protocols, results can be obtained in only a few seconds instead of hours or days (Whitesides G. M., Nature 442, 368-373 (2006)).

Microfluidic strategies for trapping cells can be generally categorized as active contactless cell trapping and contact cell immobilization by surface treatment or hydrodynamics. The former comprises optical (Kotnala A., et al., Lab Chip 17, 2125-2134 (2017)), dielectrophoretic (Chen X. et al., Biosens. Bioelectron. 99, 416-423 (2018)), acoustic (Evander M. et al., Anal. Chem. 79, 2984-2991 (2007)), and magnetic (Winkleman A et al., Appl. Phys. Lett. 85, 2411-2413 (2004)) trapping approaches, while the latter consists of chemically driven cell attachment to surfaces and passive hydrodynamic trapping. The approaches such as contact-free active immobilization and affinity-based cell attachment provide high purity (Park M. H. et al., J. Am. Chem. Soc. 139, 2741-2749 (2017)), accuracy, and the potential of high-throughput (Kobel S., et al., Lab Chip 10, 857-863 (2010)), but are complex and costly. In addition, for tumor cells with down-regulated or lost epithelial markers, such as EpCAM, affinity-based cell attachment methods lose efficacy (Tang Y. et al., Sci. Rep. 4, 6052 (2014)). Therefore, passive hydrodynamic trapping methods have been promoted in recent years.

Micropost arrays, microfiltration, microwell, and chamber trapping are the four main categories of hydrodynamic trapping. Compared with active microfluidic methods, hydrodynamic strategies are much simpler and offer higher throughput and lower cost ((Skelley A M et al., Nat Methods. 2009 February; 6(2): 147-152); Luo T., et al., Micromachines 10, 104 (2019)). These passive techniques rely on inherent hydrodynamic forces and channel geometries and other physical obstacles in the channels (Bhagat A. A. S. et al., Med. Biol. Eng. Comput. 48, 999-1014 (2010)). These strategies may also allow for high cell viability, mature chip fabrication crafts, easy operation, and integration with other downstream analysis.

Existing microfluidic chips with cell traps for pairing and fusion were only suitable for up to several thousand cells: Dura and colleagues describes a microfluidic cell pairing device capable of sequential trapping and pairing of hundreds of cells using passive hydrodynamics and flow-induced deformation (Dura, et al., Lab on a Chip. 14:15 (2014)); and a microfluidics-based cell-cell interaction assay that allows defined generation, real-time imaging, and longitudinal assay of lymphocyte interactions, thereby permitting direct correlative studies within each single cell (Dura, et al., Proc Natl Acad Sci USA. 2016 Jun. 28; 113(26):E3599-608). Further, technical restraints in the efficiency and throughput limit the efficacy of existing devices. In particular, there is a need for devices that require reduced complexity in operation and fabrication to facilitate high-throughput cell pairing and fusion.

Therefore, it is an object of the invention to provide systems and methods for efficient and specific cell fusion.

It is a further object of the invention to provide methods for high-throughput, low volume cell pairing and fusion to enable the scaled-up production for clinical applications.

It is a further object of the invention to provide an efficient and high-throughput fusion of different cell types for various bio-applications, particularly for personalized cancer vaccines.

### SUMMARY OF THE INVENTION

Microfluidic chips with cell traps for high throughput pairing of different cell types to efficiently generate specific hybrid cell phenotypes have been developed. The microfluidic system for cell pairing and cell fusion includes i) a transparent layer formed of any moldable, biocompatible and transparent material, preferably in a disc shape; and ii) a transparent support layer formed of a material such as a plastic, silicon or glass substrate with conductive electrodes (for cell electroporation or and cell-cell electrofusion) or without electrodes (for chemical cell-cell fusion or cell-cell interactions monitoring). Typically, the transparent layer includes a plurality of cell traps and at least one inlet port for receiving a sample of liquid and cells in the center; and the cell traps are arranged in one or more concentric circular arrays around the central inlet port forming a ring of a circular cell trap zone. In some embodiments, the area between the inlet port and the first circular array of cell traps includes a plurality of support pillars and/or a plurality of cell filters, preferably arranged in one or more concentric circular arrays around the central inlet port. A typical cell trap incudes i) a single-cell capture cup with a constriction, ii) a double-cell capture cup, and iii) one or more support pillars in contact with the support layer. Generally, the opening of the constriction is similar or smaller than the diameter of a single cell to ensure capture of only one cell, and the support pillars are placed on both sides of the trap to increase the fractional flow rate through the traps. In some embodiments, the transparent layer has a diameter between about 2 cm and about 10 cm, inclusive, preferably between about 3 cm and about 8 cm, inclusive. In some embodiments, the ring of the circular cell trap zone has an inner diameter between about 2 cm and about 4 cm, inclusive, and an outer diameter between about 4 cm and about 8 cm, inclusive. In preferred embodiments, the microfluidic system has a total number of cell traps between about 100,000 and 1,000,000, inclusive, preferably, between about 250,000 and 500,000, inclusive. In some embodiments, the transparent layer and the cell traps are made from polydimethylsiloxane. In some embodiments, the support layer is made from glass or plastic, optionally with electrodes. In some embodiments, the microfluidic system further includes a plastic cover attached to the support layer to create a reservoir for collection of cells and reagents passing through the chip. In some embodiments, the inlet port is connected to a robotic pipetting system for receiving a sample of liquid and cells. In other embodiments, the inlet port is connected to a rotary union for receiving a sample of liquid and cells and removing cells and reagents passing through the chip.

Methods of using the microfluidic chips and systems are also provided. The methods are particularly suited for pairing and fusing specific cells. A typical method of pairing and fusing cells includes the step of loading a first cell population onto the microfluidic system, followed by loading a second cell population onto the microfluidic system, so that the cells of the first population fuse with cells of the second population. The microfluidic system is generally primed prior to the loading of cells and reagents, for example, by washing and filling the microfluidic system with cell culture media and reducing air bubbles in the microfluidic system. In preferred embodiments, loading the first cell population results in a single cell of the first cell population inside the double-cell capture cup, and the loading of the second cell population results in a single cell of the second cell population inside the same double-cell capture cup. In some embodiments, the cell fusion is electrically or chemically induced, for example, via polyethylene glycol (PEG)-mediated cell fusion. Typically, the methods also involve the step of collecting the fused cells from the microfluid chip. In some embodiments, the cell fusion is between a dendritic cell and a tumor cell. Exemplary the tumor cell is derived from renal cell carcinoma, prostate, or breast carcinoma. In some embodiments, the dendritic cell and the tumor cell are autologous, and/or allogeneic. In preferred embodiments, the methods further include the step of administering the fused cell of the dendritic cells and the tumor cells to a subject in need thereof. In other embodiments, a B cell and a myeloma cell are fuse to make a hybridoma.

### BRIEF DESCRIPTION OF THE DRAWINGS

FIGS. 1A and 1B are schematics showing random and low-efficient cell fusion in conventional tissue culture systems (FIG. 1A), and efficient generation of pairs of specific cell types within a microfluidic chip (FIG. 1B).

FIGS. 2A-2C are schematic views showing the relative dimensions of an exemplary microfluidic chip. FIG. 2A is a side view showing a transparent layer in the shape of a disc with a diameter of about 4 cm and a height about 5 mm, including an inlet port in the center, pillars, cell filters, and cell traps arranged in a concentric fashion around the center inlet port, and a support glass layer. FIG. 2B is a top view showing the transparent layer in the shape of a disc with a diameter of about 4 cm with central circular area of pillars and cell filters, and an outer ring of cell traps (trap zone). FIG. 2C is an enlarged top view of the areas boxed in FIG. 2A and FIG. 2B showing rows of pillars and cell filters closer to the center of the transparent layer and rows of cell traps, all arranged in a concentric fashion.

FIGS. 3A-3C are views of an exemplary cell trap (1000). FIG. 3A is a top view of the cell trap (1000) showing different components including 1100 forming the back of a double cell trap, 1200a and 1200b are two front side portions forming a double cell trap with 1100. A single cell trap is formed with 110a and 110b, with a constriction similar/smaller than single cell diameter to ensure capture of only one cell. Openings 120a and 120b allow direction of flow to bring cells into the trap. D: Depth; W: width, and Const: constriction. In an exemplary cell trap, the depth is about 21 microns and width about 10 microns. In an exemplary chip, the row spacing between cell traps is about 26 microns, and column spacing about 14-18 microns. A single cell trap is formed with 110a and 110b, with a constriction formed by 112a and 112b. FIG. 3B is a side view of the cell trap. FIG. 3C is another side view of the cell trap. Openings 120a and 120b allow direction of flow to bring cells into the trap. 122a and 122b are openings on the support to allow outward flow (from top to bottom) enables deformation/squeezing of the cells to transfer them from single-cell to double-cell traps (applying outward flow in the chip). 110a and 110b have a concave shape to provide support to cells captured inside the double cell trap.

FIGS. 4A-4B are schematics showing enlarged side views of an exemplary inlet port in the center of the transparent layer along with a support glass layer. FIG. 4A shows the directional flow when cells and reagents are loaded into the chip. FIG. 4B shows the directions of the fluid flow and centrifugal force during spinning.

FIG. 5 shows an exemplary rectangular glass slide and exemplary square glass substate with electrodes as a support layer; an exemplary silicon disc with inlet port, an exemplary silicon disc with central chamber, and an exemplary plastic cover with opening on top.

FIGS. 6A-6C are schematics showing direction of liquid flow and forces for cell pairing and fused-cell collection in the chips. Outward liquid flow creates shear forces that push the cells into the traps (FIG. 6A). In stationary chip, inward liquid flow (created by syringe pulling) release the cells from traps to collect them into the syringe (FIG. 6B). Using high density media and spinning the chip at high speed create buoyant force that pushes the cells inwardly to transfer them to the central chamber (FIG. 6C).

FIGS. 7A-7F are schematics showing steps of cell pairing and fusion. Initial loading of samples including first population of cells brings these cells into single-cell traps (FIG. 7A); outward flow enables deformation/squeezing of these cells to transfer from single-cell traps to double-cell traps (FIG. 7B); loading of another population of cells brings these cells into single cell traps (FIG. 7C); outward flow enables deformation/squeezing of the cells of the second population to transfer from single-cell traps to double-cell traps to achieve cell paring of these two different cell types (FIG. 7D); electrical or chemical fusion method applies to allow fusion of these two cells (FIG. 7E); and fused cells are still in double-cell traps ready for collection (FIG. 7F).

FIGS. 8A-8E are top and side views of the microfluidic chip under different forces once samples are loaded including fluid flow, centrifugal force, and/or buoyant force. FIG. 8A and FIG. 8B are a side view and a top view, respectively, showing that equalizing liquid levels on both sides of the transparent layer (forming hydrodynamic equilibrium) prevents liquid flow during spinning and allows sedimentation of the cells towards traps at relatively constant speed (terminal velocity). FIG. 8C and FIG. 8D are a side view and a top view, respectively, showing that spinning the chip at high speeds creates liquid flow for washing and cell deformation/trapping steps. FIG. 8E shows large buoyant force when a liquid with a high density is being introduced to the spinning chip. Elevating the rotational frequency increases the buoyant force applied on the cells, which is against the direction of the centrifugal force and releases the cells from the traps to travel to the central chamber for collection.

FIGS. 9A-9D are set-ups of exemplary stationary chips. FIG. 9A and FIG. 9B are a top view and a side view, respectively, of an exemplary stationary chip having a disc shape transparent layer with a fluid inlet port in the center for loading of samples. FIG. 9C and FIG. 9D are a top view and a side view, respectively, of a previously reported 900-cell trap chip (Dura B., et al., Lab on a Chip. 14:15 (2014)), which contains a rectangle channel or trap zone, and utilizes different ways to transfer liquid and cells into the chip.

FIGS. 10A-10C are exemplary chips of various sizes. Depending on the cell trap sizes, up to 400 traps can be positioned in every 1 mm² area of the cell trap ring-zone. In this embodiment, the constriction sizes are not a fixed value, and increases at traps located in larger circular arrays or radii. Exemplary 5.7 cm disc has 1.7 cm thick cell trap ring where the constriction size of cell traps at radii of 1.2, 2, and 2.8 cm are 5, 5.5 and 6.5, μm respectively. FIG. 10A shows an exemplary chip with 294,000 cell traps with a diameter of about 4 cm and 8.5 mm thickness of cell trap ring or trap zone. FIG. 10B shows an exemplary chip having increased number of traps, while maintaining the thickness of the trap-zone ring constant. For example, disc diameter is 5.7 cm, while the thickness of cell trap ring or trap zone remains the same and is 8.5 mm, respectively. FIG. 10C shows an exemplary chip having a larger cell trap zone and increased size of constriction (FIG. 3B or 3C 112a) between single-cell and double-cell traps at larger circular arrays. FIG. 10D shows the constriction sites for double and single cell traps, with support pillars.

FIG. 11 is a side view of an exemplary system including a high throughput cell pairing and fusion chip, and a robot pipetting system for automated (contact-less) delivery of cell solution and reagents. A spindle motor required for controlling the rotational frequency of the chip is not shown in the figure.

FIG. 12 is a schematic side view of an exemplary hybrid chip with rotary-union that can use both mechanically and centrifugally induced forces for automated cell manipulation and collection.

### DETAILED DESCRIPTION OF THE INVENTION

### I. Definitions

The “microfluidic devices” or “microfluidic chips” allows fluid flow in the micro-liter and nano-liter scale in precisely defined geometries and facilitates simultaneous manipulation and analysis at a single cell level on fully integrated and automated chips. In some embodiments, such devices have one or more inlets and outlets for independent flow as well as for seeding or infusing cells into a ready-to-use device. The devices may allow for control of conditions such as media flow and shear stress to propel cells towards cell traps, temporarily deforming the cells to transfer them from single-cell to double-cell traps, and minimize damage to the cells. The devices may be arrayed and packaged in different format (often circular format) to perform cell-cell interaction assays or cell fusion screening. In various embodiments, the devices are configured to be fitted (or are fitted) with biosensors to monitor environmental conditions such as O₂, CO₂, flow rates, pH, and temperature.

The term “cell pairing” means bringing specific cells into contact for purposes such as studying cell-cell interaction or for cell fusion. In most current cell-pairing techniques, individual cells are first trapped, and then two trapped cells are paired with each other. Generally, the disclosed microfluidic chip allows cell-pairing at the single-cell level, particularly in large quantities, such as quantities above 10,000 paired cells.

The term “cell fusion” or “cell-cell fusion” is a dynamic biological phenomenon, which plays an important role in various physiological processes, such as tissue regeneration. In some embodiments, the cell fusion is carried out ex vivo or in vitro to provide fused cells with desirable properties. In some embodiments, the cells to be fused are dendritic cells and tumor cells for providing cancer immunotherapy. In other embodiments, the cells to be fused are antibody-secreting B cells and myeloma cells for generating hybridoma.

The term “cell trap” means a device shaped as a receptacle or cup for single-cell capture. The cup is typically sized to have a width similar or larger than the mean diameter of exemplary eukaryotic cells. In some forms the trap includes an opening that is sized similar to or smaller than a single cell diameter, to ensure capture of only one cell within the trap.

Use of the term “about” is intended to describe values either above or below the stated value in a range of approx. +/−10%; in other embodiments the values may range in value either above or below the stated value in a range of approx. +/−5.

### II. Chip

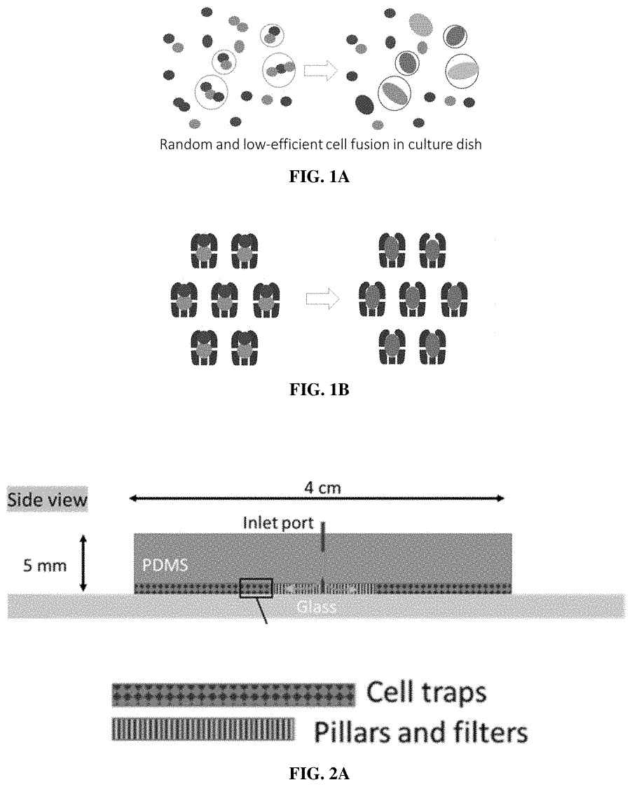

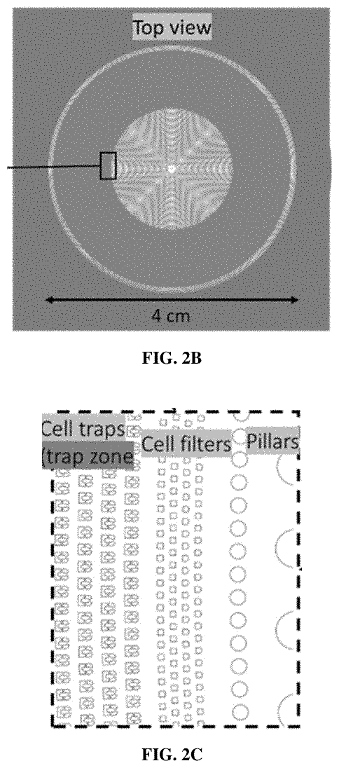

Devices for cell pairing and fusion are provided. The devices include chips that facilitate cell pairing and fusion within a single component. Generally, the chips are fabricated from transparent materials such as deformable silicon (Polydimethylsiloxane, PDMS) and contain concentric circular arrays of cell traps. The chips also include a support layer, such as glass, and, optionally, electrodes for cell-cell electrofusion. Without electrodes they can be used for chemical- (e.g., PEG-) based fusion of the paired cells. The chips solve the problems of random and inefficient cell fusion existing in conventional processes, which are due to random distribution of cells in a dish/tube/cuvette (FIGS. 1A-1B). A schematic representation of the structure and function of the chip is provided in FIGS. 2A-2C.

Typically, the chips include a silicon (e.g., polydimethyl siloxane, PDMS) layer and a support layer (e.g., glass or plastic). Typically, the glass or plastic support layer is bound to the transparent layer. The number, geometry and sizes of the traps can be adjusted for different cell pairing/fusion applications.

Other suitable materials for fabricating one or more components of the microfluidic chips include inorganic materials including glass, silica, silicon, metal, or the like or plastics or polymers, including dendritic polymers, such as poly(vinyl chloride), poly(vinyl alcohol), poly(methyl methacrylate), poly(vinyl acetate-maleic anhydride), poly(dimethylsiloxane)monomethacrylate, cyclic olefin polymers and copolymers including copolymers of norbornene and ethylene, fluorocarbon polymers, polystyrenes, polypropylene, polyethyleneimine; copolymers such as poly(vinyl acetate-co-maleic anhydride), poly(styrene-co-maleic anhydride), polysaccharide, polysaccharide peptide, poly(ethylene-co-acrylic acid) or derivatives of these or the like. The materials for forming the devices or components or parts thereof may be selected based on desired mechanical, cell-interacting, or other properties for optimizing cell culture for distinct types of cells.

A. Cell Trap Layer

The cell trap layer is typically in a disc shape having a diameter of about 2-20 cm, preferably 3-10 cm, more preferably 4-7 cm. Often, in prototyping stage, transparent biocompatible silicon (Polydimethylsiloxane, PDMS) that is patterned via soft-lipography process is used as the cell trap layer. Alternatives materials for mass manufacturing include injection-moldable biocompatible plastics. Any moldable, biocompatible and preferably transparent material could be used. Examples include plastics such as polymethylmethacrylate (PMMA) or polystyrene used in conventional cell culture dish.

The cell trap layer typically contains concentric circular arrays of large pillars, cell filters, and cell traps (trap zone ring) (FIGS. 2A-2C).

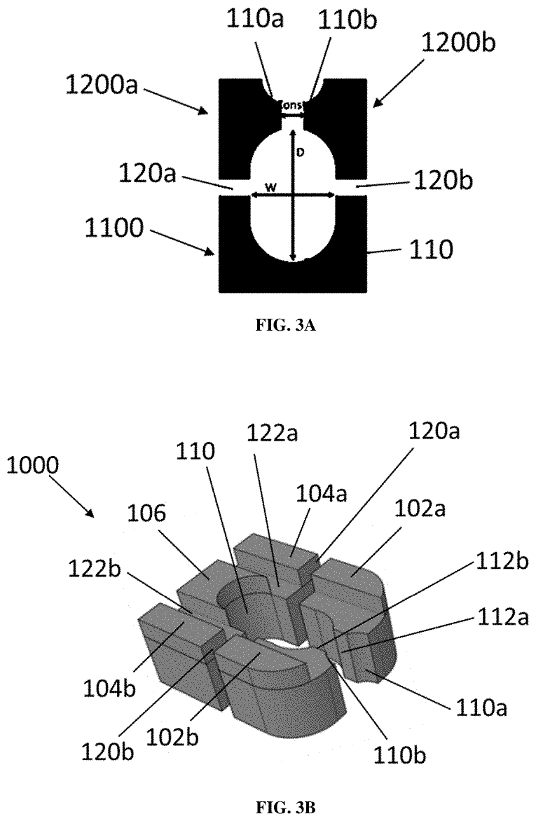

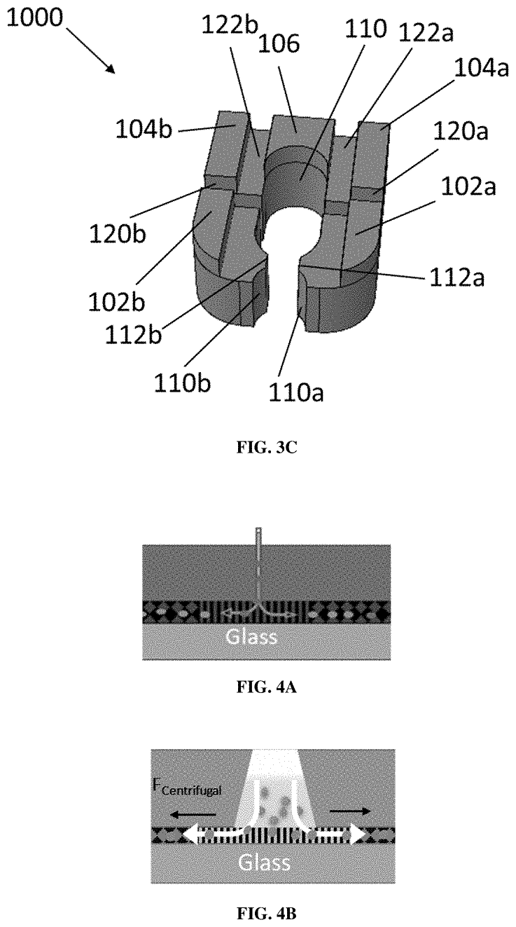

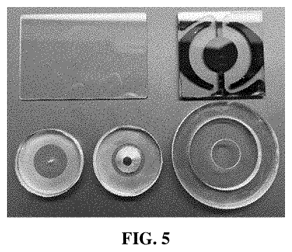

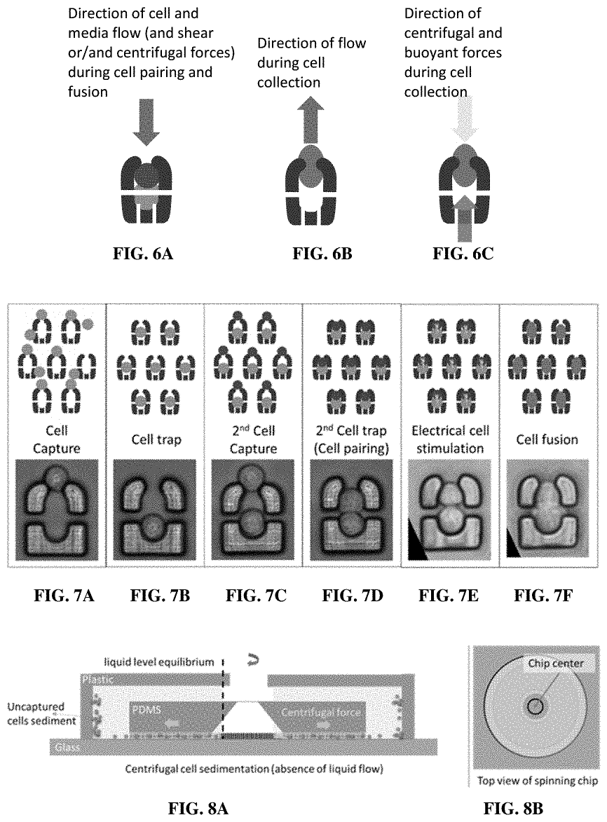

The cell traps on the microfluidic chip are suitable for cell types with a wide size range of cell diameters (±30% average cell size), for example between from about 7 μm and about 13 μm, inclusive, for example, about 7, 8, 9, 10, 11, 12 or about 13 μm (FIGS. 3A-3C). For a larger range of cell sizes, the cell trap dimensions are scaled up accordingly. Instead of changing cell trap size, it is also possible to use iso-osmolar and hypo-osmolar buffers to, respectively, shrink and enlarge the cells of different sizes.

The microfluidic chip has weir-based hydrodynamic cell traps densely arrayed within a flow-through channel bonded to a support layer. Each cell trap contains a two-cell trap connected to a single-cell trap via a narrow constriction, and its structure is adjusted to maximize the ratio of the fluid flow into the cups versus around the cups (FIGS. 3A-3C). Support pillars are placed around the capture cups (sides and back) to provide fluid flow through them for guiding cells into the traps for capture.

In one embodiment, the transparent layer made from PDMS is a silicon disc with a diameter of about 4 cm and contains up to 294,000 cell traps that are arranged in up to 144 concentric circular arrays within an 8.5 mm thick ring having an inner radius of 10 mm. In some embodiments, the number of traps in each array increases after every 6th array to maintain the horizontal gap between cell traps similar along the increasing chip radius/circumference. For example, in some embodiments, each of the first 6 circular arrays contains 1400 cell traps while each of the last six arrays contains 2770 traps.

1. Cell Traps

The transparent layer includes one or more cell traps. Typically, the structure of a trap 1000 includes (i) a single-cell capture cup (formed with 110a and 110b of FIGS. 3A-3C) with a constriction (formed by 112a and 112b of FIGS. 3A-3C) similar to or smaller than single cell diameter to ensure capture of only one cell, and (ii) a double-cell capture cup with width similar or larger than the mean diameter of cell populations.

In some embodiments, the depth of the double-cell capture cup is similar to the sum of diameters of cells that will be paired inside a single capture cup. The single-cell capture cup is sized to capture only one cell and the two-cell capture cup is tailored to accommodate cell pairs with proper alignment within the traps. Support pillars on both sides of the trap (and openings in each side) increase the fractional flow rate through the traps (see FIGS. 3A-3C).

In general, the 3D geometry of cell traps is especially designed to ensure liquid can flow through traps from both directions; (FIGS. 3A-3C) outward flow (from top to bottom) enables deformation/squeezing of the cells to transfer them from single-cell capture cup to double-cell capture cup (applying outward flow in the chip), and inward flow allows final collection of fusion cells.

It has been established that narrow openings at the sidewalls of the cell traps provide additional flow paths, and importantly help maintain similar flow resistances (therefore similar fluidic patterns) of the capture cups after the loading of the first cell population (that is, the flow resistance of the capture cups does not increase significantly due to the presence of trapped cells). This in turn facilitates sequential capture of the second cell population and their transfer from the single-cell traps to two-cell traps with similar fluidic operation. Therefore, in some embodiments, the cell traps include one or more openings within one or more of the sidewalls. For example, in some embodiments, the cell traps include a multiplicity of opening within the sidewalls, for example, in the middle of the sidewalls.

Typically, the openings in the middle of sidewalls are smaller than the cell radius to prevent cells from squeezing out of the traps, especially with increased flow rates during the cell deformation/trapping step. Therefore, in some embodiments, the openings are smaller than or equal to 9 μm, for example, 8, 7, 6, 5, 4, 3, 2 or 1 μm, or smaller than 1 μm, for example, 0.1 μm.

A support pillar positioned at the rear of the double-cell capture cup ensures cell entrapment once the cells are in the cups. The horizontal and vertical gaps as well as trap (and reservoir) height are greater than the largest cell diameter. Therefore, in some embodiments, the height of one or more of the vertical gap, trap and reservoir is greater than or equal to 9 μm, for example, 10, 15, 20, 25, 30, 35, 40 or 50 μm, or greater than 50 μm, for example, 100 μm. It has been established that curvatures at the top-edge of the traps reduce the risk of cells getting stuck between the gaps.

In some embodiments, the support layer contains more than 100, more than 1000, more than 100,000 or more than 500,00 cell traps. In an exemplary embodiment, the support layer includes 294,000 cell traps.

i. Concentric Arrays of Cell Traps

Typically, a multiplicity of cell traps is arranged as a concentric arrangement of circular arrays. The number of arrays is from between 10 and 1000, for example, between 20 and 200, for example, between 100 and 150, disposed in a concentric circular manner Each of the cell traps within an array are positioned with proper spacing relative to one another to minimize clogging and at the same time maximize cell capture. All three: i, the horizontal space between cell traps in a circular array, ii. the vertical space between parallel circular arrays, and iii, channel height (cell trap zone thickness) are greater than the largest cells; for example, typically are between 1.1 and three times greater than the average size of the largest cell type.

It has been established that concentric arrangement of cell traps enables integrating large numbers of traps for high-throughput cell pairing and fusion, for example, using centrifugally induced forces which provide small pressure gradients among traps in a large ring of trap-zone. In preferred embodiments the cell traps are arranged in a circular configuration. The circular arrangement of cell traps allows for having low gradient of forces between first and last trap in a large trap zone area. It has also been established that increasing the area of the cell trap zone ring, while maintaining an equal thickness of the ring small or/and gradually increasing the size of single-cell trap constriction allows making larger chips for the manipulation of even more cells. In exemplary embodiments, the thickness of the ring is from about 1 mm to about 50 mm, for example, 2 mm to 120 mm, preferably 8.5 mm. The ring has an inner radius of 100 μm to about 100 mm, inclusive, for example, between 1 mm and 20 mm, for example, 10 mm. In some embodiments, each of the first one to ten circular arrays contains between 100 and 10,000 cell traps, more preferably, between 1000 and 5000 cell traps. In an exemplary embodiment, each of the first 6six circular arrays contains 1400 cell traps.

In some embodiments, each of the last 1 to 10 circular arrays contains between 200 and 20,000 cell traps, for example, between 2,000 and 8,000 cell traps. In an exemplary embodiment, each of the last six arrays contains 2770 traps. The size of the cell traps may vary from a size such as 25 to 150 μm depending on cell diameter (5-100 μm) and the number of cells (1, 2, 3 and 4 cells) intended to be paired inside each trap. For example, for pairing two cells of 10 μm, about 375 traps can be designed/impeded in every 1 mm² area of the trap zone.

2. Inlet Port

The transparent layer typically has at least one inlet port for receiving a sample of liquid and cells. Exemplary inlet ports are shown in FIGS. 4A-4B. In preferred embodiments, the oner or more inlet port is located at or near to the center of the transparent layer. The diameter of an inlet port is sized to permit passage of cells and other reagents, and therefore is always larger than cell diameter, and typically tighter than the outer diameter of a syringe-needle or connector (for example, smaller than 0.718 mm for syringe needle 22G). The needle typically is used to connect a disc to an external sources of cells or reagents. Example of reservoirs used as external liquid sources are syringe on a mechanical-pumps (syringe pumps) and bottle/tube connected to peristaltic pump. For example, in an exemplary embodiment, an inlet port has a diameter of between 20 μm to about 500,000 μm, inclusive.

3. Internal Support Pillar and Cell Filters

In some embodiments, the area between the center of the chip and the first circular array of cell traps includes one or more internal support structures. Exemplary positioning of these internal support structures is shown in FIG. 2C. It has been established that, in the absence of support structures within the transparent layer, the surfaces of the transparent layer in areas without cell traps or filters will bind to the support surface due to collapse of the silicon wall, and the liquid pathway becomes blocked. Therefore, in some embodiments, the transparent layer of the chip includes one or more support structures. In some embodiments, the support structures are circular pillars. The circular pillars have a diameter from about 10 μm to about 1000 μm in diameter. In a preferred embodiment the circular pillars have a diameter of about 100 μm.

In some embodiments the chips include one or more cell filters. It has been established that cell filters help separating or stopping cell clamps and debris before they reach the cell trap zone. The cell filters typically have a diameter from about 10 μm to about 100 μm in diameter, with gaps smaller than double the size of the cell diameter. In a preferred embodiment the cell filters have a diameter of about 17 μm. In some embodiments, the PDMS layer contains similar structures of cell filters and pillars at the outer edge of the chip, i.e., beyond the arrays of cell trap (trap zone) away from the center inlet.

B. Support Layer

The chip typically includes a support layer attached to the transparent layer. The support layer can be made out of glass, plastic or silicon.

1. Support Layer with Electrode

Some exemplary support layers are shown in FIG. 5. In some cases where electrofusion is required, a substrate with conductive electrodes is used as the support layer. The outer electrode is usually located right after the last circular array, over several cell filters or and support pillars.

In other embodiments, the support layer is designed to not contain any electrodes. The chips with or without electrodes can be used for cell pairing and chemical-based cell-cell fusion.

C. Cover

In some embodiments, the chip also includes a cover or sheath. The cover or sheath is used to create a reservoir for collection and/or protection and retention of cells and liquids passing through the chip. In some embodiments the cover or sheath is formed of a material that insulates the cell from one or more of external dirt and debris, contamination, heat, electrical charge, sunlight, and water. An exemplary cover is formed from plastic. Typically, the cover encapsulates the entire surface of the chip and typically includes one or more access port for administering cells and reagents into the chip.

### III. Configuration of Chip

Based on the methods and forces used in the process of cell pairing and collection, the chips are categorized as either (i) stationary; or (ii) spinning.

Exemplary large stationary and spinning microfluidic chips with 294,000 cell traps for pairing of different cell types to efficiently generate specific hybrid cell phenotypes are described. In some embodiments, the microfluidic chip includes more than 300,000 cell traps, for example, more than 400,000 cell traps, more than 500,000 cell traps, more than 600,000 cell straps, more than 700,000 cell traps, more than 800,000 cell traps, more than 900,000 cell traps, and more than 1 million cell traps. The chips could contain less cell traps, 100,000 cell traps or 50,000 cell traps.

A. Spinning Chips

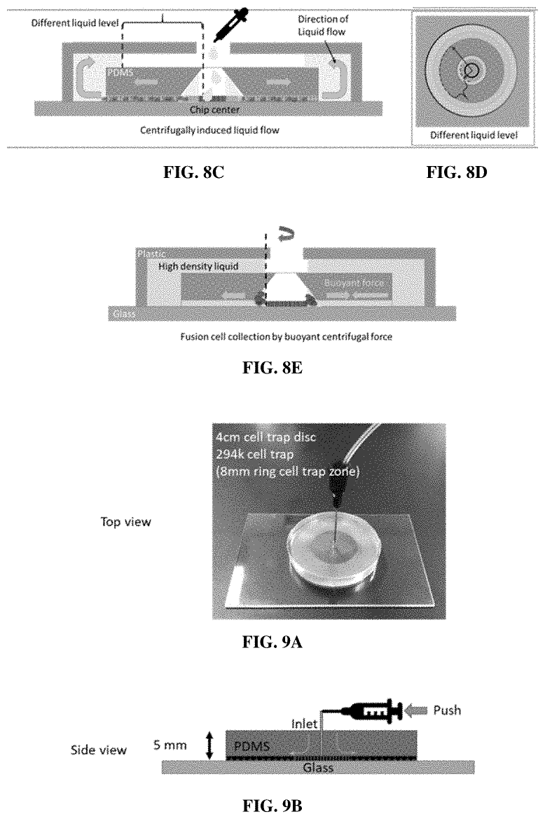

In some embodiments, the chips are configured as spinning chips, such as those shown in FIGS. 8A-8E. The spinning chip enables the use of centrifugally induced forces and liquid flow for cell pairing and fused-cell collection. In an exemplary embodiment, a plastic cover such as the one depicted in FIGS. 8A-8E can be attached to the support layer to create a reservoir for collection/accommodation of cells and reagents passing through the chip. In other embodiments, the plastic cover is replaced by channels or chambers of different design.

As shown in FIGS. 8A-8E, the centrifugation causes the uncaptured cells to sediment to the outside. In one embodiment shown in FIG. 8E, high density liquid is used collect cells as they accumulate on the top of the high density liquid.

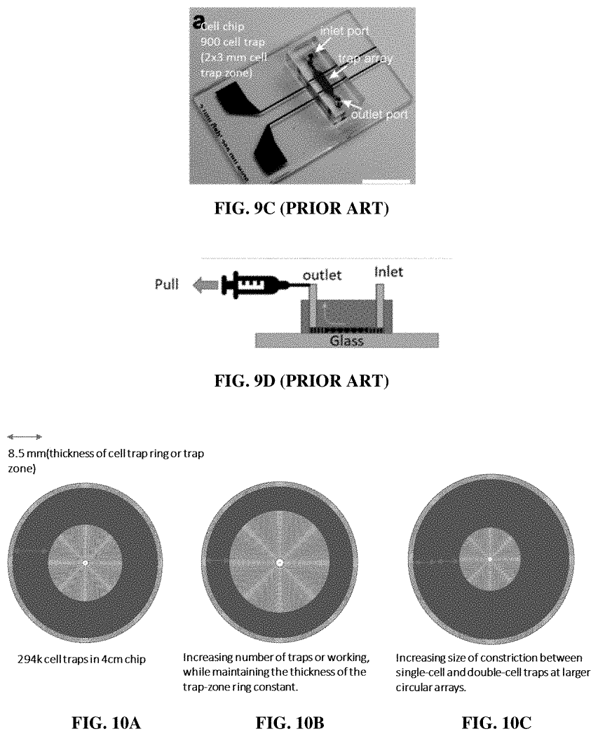

In comparison to previous microfluidic chips such as those shown in FIGS. 9C-9D, these chips are larger, and significantly increase the throughput of cell pairing and fusion, for example 10-fold, 20-fold, 30-fold, 40-fold, 50-fold, 60-fold, 70-fold, 80-fold, 90-fold, 100-fold, or more than 100-fold relative to prior microfluidics chips such as those described by Dura, B., Liu, Y. & Voldman. J. (2014). Deformability-based microfluidic cell pairing and fusion. Lab on a Chip, 14(15), 2783-2790. In the earlier described microfluidic chips, the traps are arranged in parallel lines instead of parallel circular arrays as described herein. These contained up to 1500 traps and could be scaled up to contain 10,000 traps.

One type of spinning chips contains a funnel-shaped central chamber in which cells and reagents are directly pipetted into, and fused cells are collected at the end. In the spinning type of the chip, centrifugally induced forces and liquid flow are employed for cell pairing and collection.

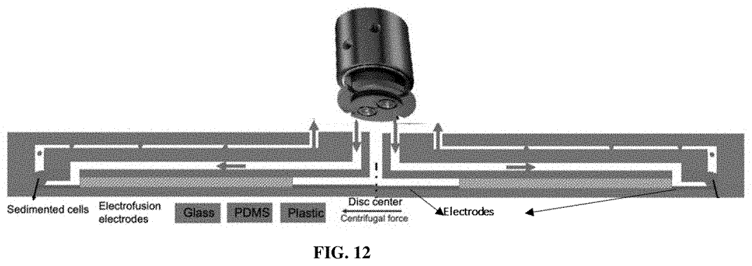

Other types of spinning chips are similar to stationary disc shown in FIG. 9A, or the automated system shown in FIG. 12. Cell manipulation using both centrifugally induced forces and liquid flow (hydrodynamic forces) provides many advantages, including high-efficiency cell capturing via cell sedimentation at terminal velocity; employing centrifugally induced liquid flow for cell deformation/trapping; employing centrifugal buoyancy force to release cells from traps and transfer them to a central chamber for their collection; removal of air bubbles with centrifugal-buoyancy and inertial forces; contactless cell/liquid delivery and extraction; and sedimentation of uncaptured cells at outer rim of the chip for their collection after cell capture step. The spinning chip is designed to exploit each of these features for optimal cell merging and pairing.

1. High-Efficiency Cell Capturing Via Cell Sedimentation at Terminal Velocity.

The spinning chip is designed to facilitate high-efficiency cell capturing via cell sedimentation at terminal velocity. On the spinning chip, cells in the absence of liquid flow travel at a constant speed, i.e., terminal velocity. The sedimentation of the cells directly towards the cell trap opening is expected to increase cell capturing efficiency when compared with the flow-based methods in which cells are propelled both towards and around the traps (FIG. 8A).

2. Employing Centrifugally Induced Liquid Flow for Cell Deformation/Trapping.

The spinning chip is designed to exploit centrifugally induced liquid flow for cell deformation/trapping. In the spinning chip, both centrifugal force from spinning and shear forces from liquid flow are employed to deform the cells into a double-cell capture cup (FIG. 8C). When liquid is injected to the chamber and the chip is spun at high speeds (e.g., more than 10 Hz, or 4 Relative Centrifugal Force (RFC)), the centrifugal force drags the liquid toward the outer periphery and the induced liquid flow and shear force is used for cell deformation/trapping step. The spinning speed defines the amount of liquid flow and centrifugal forces acting on the cells. The amount of force may vary between 4 and 200 RFC.

3. Employing Centrifugal Buoyancy Force to Release Cells from Traps and Transfer them to a Central Chamber for their Collection.

The spinning chip is configured to utilize centrifugal buoyancy force to release cells from traps and transfer them to a central chamber for their collection. In some embodiments, cell collection is achieved using a high-density liquid and spinning the chip at high speed (e.g., 40-50 hz) to create adequate centrifugal buoyancy force to release and push the cells towards the central chamber. When a liquid with a high density is being introduced to the spinning chip, elevating the rotational frequency increases the buoyant force applied on the cells, which is against the direction of the centrifugal force (FIG. 8E). Creating adequate cell buoyant force allows trapped cells (fused cells) to deform or squeeze-out of the traps and travel to the central chamber for collection. Any commercial high density cell culture media or biocompatible buffer (such as a density gradient medium) can be used, or cell media density can be increased by adding additional fetal bovine serum (FBS) or other medium that will not cause the cells to lyse.

4. Removal of Air Bubbles with Centrifugal-Buoyancy and Inertial Forces.

The spinning chip is configured to remove air bubbles with centrifugal-buoyancy and inertial forces. The risk of air bubble formation in the chip (e.g., between and inside traps) during the initial wetting of the chip (even with ethanol) increases proportionally with the chip size and pre-storage duration. This is because of gradual hydrophobic recovery (Hydrophilic loss) after PDMS and Glass plasma binding.

In the spinning chip, centrifugal buoyancy force and inertial forces (from alternating the chip spinning speed) are employed to remove the air bubbles from the surfaces (cell trap zone) and drive them towards the chip center. Alternating the chip spin speed between a low (e.g., 15-25 Hz) and a high (e.g., 30-50 Hz) rotational frequency creates adequate buoyant force to push the air bubble towards the chip center. Inertial forces also facilitate removal of air bubbles drifting between the traps. The duration required for air bubble removal varies (e.g., 20-60 sec) depending on the spin alternation speeds and acceleration used.

5. Contactless Cell/Liquid Delivery and Extraction

The spinning chip is designed to enable contactless cell/liquid delivery and extraction. The central chamber on the spinning chip is used for direct injection of cells and reagents, and final collection of fused cells. Employing centrifugal force and central chamber enable contactless liquid pipetting and collection manually or automated by a robotic pipette as shown in the schematic in FIG. 11. For example, in some embodiments, at the top of the PDMS layer, a vacuum/suction nozzle continuously collects waste solutions from the outlet ring. This avoids the need for tube connection and syringe pumps to avoid risk of air bubble introduction.

6. Sedimentation of Uncaptured Cells at Outer Rim of the Chip for their Collection after Cell Capture Step.

As shown in FIG. 8A-8E, the spinning chip is designed to sediment uncaptured cells at outer rim of the chip for their collection after cell capture step. In the spinning chip, cells that are not captured by cell traps are sedimented at the outer rim of the chip and can be collected right after the cell capture step. While the cell sedimentation at the outer rim occurs, the waste solution continuously flows to the upper chip layer towards the opening of the plastic cover and can be collected by contactless vacuum nozzle. In the hybrid chip, a channel at the outer edge of the chip can be used to collect the sedimented cells.

B. Stationary Chips

In some embodiments, the chips are configured as stationary chips.

Stationary chips contain an inlet-port that is connected to mechanical pumps (syringe pumps) for injection of cells/reagents and final withdrawal/collection of fusion cells. An exemplary set-up is shown in FIGS. 9A and 9B. Therefore, cell manipulation in this type of chips is controlled by mechanically induced liquid flow and shear forces.

C. Hybrid Chips.

In hybrid chips, the liquid inlet at the disc center, for example, a needle (similar to stationary disc FIGS. 9A and 9B) or a rotary union (FIG. 12), is connected to external liquid supplies/pumps such as syringe pumps. In the hybrid system both centrifugal force from spinning and hydrodynamic forces from externally induced liquid flow are employed to control the process of cell pairing and collection.

D. Strategies for Design of Larger Chips

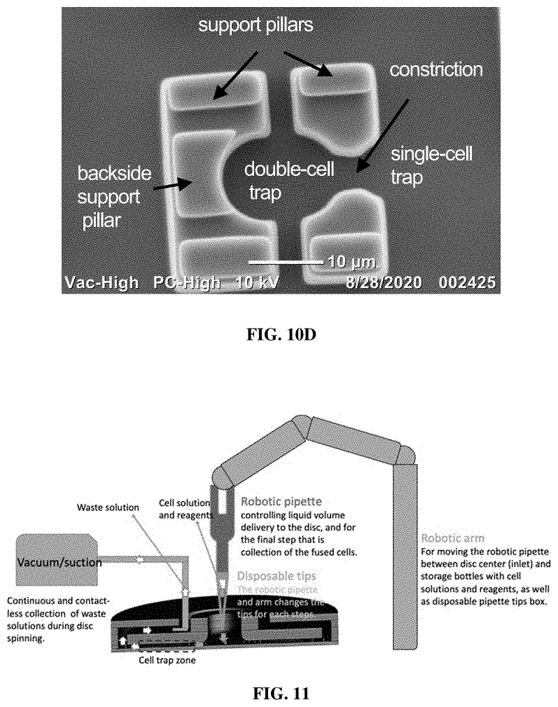

In some embodiments, the chips (FIG. 10A) are designed for combining and/or pairing cells with far greater capacity than existing chips such as those shown in FIG. 9C and FIG. 9D. Liquid flow speed is not constant and decreases as the area or number of traps in larger circular arrays increases. At constant flow rate, the gradient of forces is proportional to the radius and thickness of the trap-zone-ring, and cells near center experience greater shear forces than cells near outer periphery of the chip. Therefore, in some embodiments, larger chips with more cell traps (FIG. 10B) maintain the gradient of forces, while maintaining the smaller thickness of the trap-zone-ring, using larger working areas (or ring radius) to increase the trap area. In another embodiment, larger chips with more cell traps increase the size of constriction between single-cell capture cup and double-cell capture cup to assure cells near the outer rim of the chip require lower shear force to deform/trap (FIG. 10C). In the spinning chip, sedimentation (cells traveling at terminal velocities) facilitate cell capturing over large ring areas, and centrifugal force that is greater at larger radii also may compensate with decreasing shear force to help with cell deformation for trapping.

### IV. Systems and Kits

A. Automation

In some embodiments, the chips are loaded and processed using automated processes. For example, in some embodiments such as that shown in FIG. 11, automation of the cell pairing process uses centrifugal force and continuous and contactless collection of waste solutions during disc spinning and a robotic pipet to control liquid delivery to the disc and for collection of the fused cells. and. For automation of the cell-pairing methods, the cone-shaped central chamber is required to be smooth to avoid cell trapping in its surface.

In some forms, automation includes using a rotary union. Using a rotary union allows for developing hybrid spinning chips with inlet/outlet ports and valves. In that case moving ports of the rotary union are connected to the inlet/outlet ports of the chip, while stationary ports of the union are connected to mechanical pumps and chambers (e.g., waste collection bottle). An exemplary hybrid chip is shown in FIG. 12. Mechanically and centrifugally induced forces and liquid flow are combined for cell and reagent manipulation. For example, while spinning helps with bubble removal and may enable more efficient cell capturing and final cell collection, combining the forces can facilitate cell deformation/trapping within larger chips, having increased capacity.

B. High-Throughput Applications

In some embodiments, the microfluidic chips and systems are suitable for high-throughput design and monitoring of cell-cell interactions.

In some embodiments, the microfluidic chips and systems are suitable for high efficiency cell fusion, for example, in generating hybridoma and dendritic-tumor fusion cells etc. The microfluidic chips are particularly suited for generating specific fusion cell phenotypes based on the specific cell pairing and fusion.

C. Data Structures and Computer Control

Data structures used in, generated by, or generated from, the disclosed method generally are any form of data, information, and/or objects collected, organized, stored, and/or embodied in a composition or medium. An image showing one or more parts of the microfluidic device including the cell traps, cell filters, and fluid channels stored in electronic form, such as in RAM or on a storage disk, is a type of data structure.

The method, or any part thereof or preparation therefor, can be controlled, managed, or otherwise assisted by computer control. Such computer control can be accomplished by a computer-controlled process or method, can use and/or generate data structures, and can use a computer program. Such computer control, computer-controlled processes, data structures, and computer programs are contemplated and should be understood to be disclosed herein.

D. Kits

Kits are also disclosed. The kit can include a single microfluidic chip or multiple microfluidic chips, and instructions for using the microfluidic chips. In some embodiments, the kit also includes one or more solutions for use with the microfluidic chips, such as those used for priming and washing the microfluidic chips. In other embodiments, the kit may also contain additional components such as beads for calibrating robots, monitoring the set-up, and optimize imaging. In some embodiments, the beads are fluorescently labeled beads.

### V. Fused Cells

The microfluidic devices are particularly suited for pairing and/or fusing specific cells. Generally, the microfluidic devices are suitable for cell types with a wide size range (cell diameters ˜9-25 μm).

A. Types of Cells for Cell-Fusion

Generally, any cells that can be fused using conventional cell culture in a dish/tube/cuvette should be able to fuse on the microfluidic chips. In some embodiments, size of cell traps and cell filters and array spacing is optimized for different cell types.

In some embodiments, the cells to be loaded onto the microfluid chips for fusion are mammalian cells. In other embodiments, the cells to be loaded on to the microfluid chips for fusion are antibody-producing B cells or transformed cells.

1. Dendritic Cell and Tumor Cell Fusion

Dendritic cells (DCs) are professional antigen-presenting cells capable of initiating specific primary immune responses, including the activation of tumor-reactive cytotoxic T-cells (CTLs) (J Banchereau and R M Steinman Nature. 1998 Mar. 19; 392(6673):245-52; Kurokawa T, et al., Int J Cancer 2001; 91:749-756). Various tumor vaccination strategies have been developed based on the loading of DCs with tumor-associated antigens (TAAs) (Thurner B, et al., J Exp Med 1999; 190:1669-1678; Mackensen A, et al., Int J Cancer 2000; 86:385-392). One promising approach involves the fusion of DCs with tumor cells (Gong J, et al., Nat Med 1997; 3:558-561; Soruri A, et al., Cancer Immunol Immunother 2001; 50:307-314). This approach is based on the idea that multiple TAAs are endogenously processed and presented by MHC class I molecules, thereby stimulating tumor-specific CTLs (Boon T, van der Bruggen P. Human tumor antigens recognized by T lymphocytes. J Exp Med 1996; 183:725-729). Several mouse models and vaccination studies have shown regression of established tumors after hybrid cell application (Stuhler G, Walden P. Cancer Immunol Immunother 1994; 39:342-345; Gong J, et al., Proc Natl Acad Sci USA 1998; 95:6279-6283; Gong J, et al., Immunology 2000; 101:316-324; Kugler A, et al., Nat Med 2000; 6:332-336; Trefzer U, et al., Int J Cancer 2000; 85:618-626).

In some embodiments, the dendritic cells and tumor cells are generated by electrofusion using the chip. In some embodiments, the dendritic cells and tumor cells are generated by polyethylene glycol (PEG) using the chip. In preferred embodiments, the resulting cell population retains the functional capability of antigen-presenting cells essential for the induction of antigen-specific immune responses. In preferred embodiments, the resulting cell population is suitable for the immunotherapy of cancer.

In the clinical setting of patients with cancer, a major limitation of the use of DC-tumor fusion cells is the availability of adequate amounts of autologous tumor cells. The tumor cells are of limited availability because of the viability of the tumor samples and/or technical difficulties in the cell culture owing to the length of culture and potential contamination with bacteria and fungi. In some embodiments, the DCs are autologous DCs. In some embodiments, the tumor cells used in the DC-tumor fusion cells are autologous tumor cells. In other embodiments, the tumor cells used in the DC-tumor fusion cells are allogeneic tumor cell lines. In preferred embodiments, the fusion cells are generated with autologous DCs and allogeneic tumor cells.

In some embodiments, the tumor cells are carcinoma cell lines derived from renal cell carcinoma, prostate or breast carcinoma fused to DCs cultured from CD34+ cells. In some embodiments, the DCs are monocyte-derived DCs from the subject to be treated with the fused DC-tumor cell or from a healthy donor.

2. Cell Fusion for Generating Hybridoma

Hybridoma cells are generated via fusion between a short-lived antibody-producing B cell and an immortal myeloma cell. Each hybridoma cell constitutively expresses a large amount of one purely specific monoclonal antibody (“mAb”), and favored hybridoma clones can be cryopreserved for continuous mAb production for a long period. Hybridoma generation process takes advantage of a host animal's natural ability to generate functional, highly specific, and high affinity mAbs (Zaroff S. and Tan G. Biotechniques. 2019; 67(3):90-92). To date, several mAbs have been developed using cell fusion technology and are presently used for diagnosis, prevention, and treatment of different diseases. Exemplary hybridoma derived mAbs include Enfortumab vedotin (PADCEV®), Crizanlizumab (ADAKVEO®), Romosozumab (EVENITY®), Polatuzumab vedotin (POLIVY®), Risankizumab (SKYRIZI®), Ibalizumab (TROGARZO®), and Ravulizumab (ULTOMIRIS®).

The chips and the systems are well suited for providing a high throughput flatform to generate hybridoma. Thus, in some embodiments, the chips and the systems are for high throughput cell pairing and fusion between B cells and myeloma cells.

### VI. Methods of Uses

It has been established that chips employing arrays of cell traps can be used to enhance specific pairing and fusion of two or more cells. Methods of pairing and fusing cells include one or more steps of loading a first cell population onto a microfluidic system, followed by loading a second cell population onto the microfluidic system, and then allowing cell fusion between cells of the first population with cells of the second population. In some embodiments, the methods include one or more steps of priming the microfluidic system prior to loading of the first cell population. In some embodiments, the priming step includes washing and filling the microfluidic system with cell culture media with minimal air bubbles in the microfluidic system. Typically, loading the first cell population results in a single cell of the first cell population inside the double-cell capture cup, and the loading of the second cell population results in a single cell of the second cell population inside the same double-cell capture cup. In some embodiments, the cell fusion between the first and second population of cells is electrically or chemically induced. In other embodiments, the cell fusion between the first and second population of cells is polyethylene glycol (PEG)-mediated cell fusion. The methods optionally include one or more steps of isolating the paired cells from the chip. Therefore, in some forms, the methods include one or more steps of (i) priming the chip, (ii) loading the chip with a first and second populations of cells, (iii) pairing and fusing the cells within cell traps within the chip, and (iv) collecting the paired and fused cells from the chip.

A. Priming

The methods typically include one or more steps of priming a cell pairing microfluidic chip. At first the chip is primed according to one or more of three steps, including i) first a solvent such as ethanol is injected to the chip to remove air, then ii) cell-media is injected to remove the ethanol and to prevent cell adhesion to the chip (silicon, glass, and electrode) surfaces, and iii) bovine serum albumin (BSA) (or 2-5% Pluronic F-127 solution) is injected and incubated for a period of time that is at least 30 minutes. In some embodiments, before use, the methods include one or more steps to wash the chip one or more times with a suitable fluid for cell survival, such as ell media.

In a stationary chip, air bubbles are removed in the stationary chip by introducing a high liquid flow rate (e.g., 2-5 ml/min) and/or the PDMS area (on top of the trapped-air) is mechanically pressed to help bubbles drifting between the traps and exit the chip.

In a spinning chip, the methods prime the chip by filling the central chamber in a step-by-step manner using suitable reagents (ethanol, media and bovine serum albumin (BSA)). The methods include one or more steps to flush the priming reagents, by spinning at up to 30-50 Hz, to transfer the reagents to the trap zone. Alternating the chip spin speed with high acceleration helps drifting of any trapped air bubbles for their removal. Liquids passing the trap zone (or exiting the channel between PDMS and substrate) are accommodated in the reservoir that is formed by attached plastic cover and collected by suction pump/syringe.

B. Loading Cells

In the cell capture step, cell solution (cells in a biocompatible liquid such as media or PBS) is injected into the chip at a low flow rate (e.g., 20-40 μL/min for 20-60 sec). When cells are traveling towards the outer periphery of the chip (in all 360-degree directions), each trap captures a single cell at its single-cell trap. In the stationary chip, fractional flow rate through the traps results in cell trapping. Cells are loaded in the chamber of the spinning chip, and sedimented via spinning (at 8 to 15 Hz). Equalizing liquid levels in both sides of the transparent layer (forming hydrodynamic equilibrium) prevents liquid flow during spinning, allowing sedimentation of the cells towards traps at relatively constant speed (terminal velocity). Alternatives for cell loading in the absence of liquid level equilibrium include loading small volume of solution (5-10 μl) with high cell concentration to the source chamber and spinning the chip at a minimum speed (8-20 Hz) and integrating valves into the chip to stop or allow liquid flow during spinning.

Afterwards, the excessive cells are washed away by gradually increasing flow rate to up to 200 μL/min. The washing step in the spinning chip is carried out by filling the chamber with cell media and increasing the speed up to 15-24 Hz to induce liquid flow. After the cell capture step, applying several short pulses (3-5×2 sec) of high flow rate (2-4 ml) then results flow-induced deformation/squeezing of the cells to transfer them into the double cell capture cup. In the spinning chip, the cell trap step is carried out by filling the central chamber with media and increasing the spinning speed up to 40 Hz to generate adequate liquid flow rate for cell deformation.

Afterwards, trapping of second cells (cell pairing) is achieved by repeating the same process of cell capturing, washing and trapping steps. The trap sizes and horizontal/vertical gaps are optimized for different ranges of cell sizes, typically based on the largest cells diameter. Hypo-osmolar and iso-osmolar buffers (Dura, et al., Lab on a Chip. 14:15 (2014)) can be used to temporarily enlarge or shrink the cells in case cell diameter is smaller or much-larger than constriction between single-cell and double-cell capture cups. In chips designed for pairing murine myeloid leukemia and dendritic cells, the vertical gaps were fixed at 26 μm, while horizontal gaps, although fixed at each circular array, vary between 14-16.5 μm from one array to the next. In one embodiment, the trap depth is 21 μm, internal width is 10 μm, and constriction size is 5 μm. See Table.

C. Cells Pairing and Fusion

In some embodiments, the methods include a step of pairing and fusing the cells within cell traps within the chip.

For paired-cells electrofusion, first an electroporation buffer is injected to the chip. Depending on the type of buffer (conventional iso-osmolar or hypo-osmolar), cells may expand or shrink within 1-5 minutes. Typically hypoosmolar electroporation buffer contains [KCl 25 mM, KH₂PO₄ 0.3 mM, K₂HPO₄ 0.85 mM, and myo-inositol 90 mOsmol/kg, pH 7.2±0.2] and the iso-osmolar buffer contains identical components except that the concentration of myo-inositol is 280 mOsmol/kg. Both buffers typically have a conductivity of 3.5 μS/cm. Several pulses of high DC voltage then are applied to the electrodes. The DC voltage and number of pulses and durations vary for each cell type and fluidic design, and typically are between 1-3 kV/cm and 1-6 pulses of 20-100 μs duration. After 10 minutes the buffer is replaced with warm media and chip/cells incubated at 37° C. for 15-30 minutes. For chemical fusion of the cells, PEG-1500 is injected into the chip for 2-5 min followed by its replacement with cell media and incubation for 20-30 min. However, electrofusion is preferred because of its low toxicity to the cells and higher efficiency.

D. Collecting Fused Cells

In some embodiments, the methods include a step of collecting the paired and fused cells from the chip.

To collect the cells from a stationary chip, the direction of flow is reversed to remove the cells from the traps.

In absence of flow in spinning chip, cell collection is achieved using a high-density liquid and spinning the chip at high speed (e.g., 40-50 hz) to create adequate centrifugal buoyancy force to release and push the cells towards the central chamber. When a liquid with a high density is being introduced to the spinning chip, elevating the rotational frequency increases the buoyant force applied on the cells, which is against the direction of the centrifugal force. Creating adequate cell buoyant force allows trapped cells (fused cells) to deform or squeeze-out of the traps and travel to accommodate in the central chamber for collection.

E. Applications of the Fused Cells

In some embodiments, the methods also include a step of screening the fused cells for desired properties, optionally propagating the select fused cells for down-stream applications.

In some embodiments, the methods also include a step of administering the fused cell to a subject in need thereof.

In the case of hybridoma, the methods also include a step of identify and isolating a fused cell producing antibodies with desirable binding properties. In some embodiments, the selected clones are further dispersed into a liquid growth medium for screening and expansion.

In further embodiments, the methods also include a step of cryopreserving the fused cells.

### EXAMPLES

### Example 1: Exemplary Design for Concentric Circular Arrays of Cell Traps (in Both Stationary and Spinning Chips)

The circular arrangement of cell traps allows for low gradients of forces between the first and last trap in a large trap zone area. Increasing the area of cell trap zone ring, while maintaining the thickness of the ring or/and gradually increasing the size of single-cell trap constriction, allows larger chips to be made for the manipulation of even more cells.

Rapid Removal of Air Bubbles with Centrifugal-Buoyancy and Inertial Forces.

The risk of air bubble formation in the chip (e.g., between and inside traps) during the initial wetting of the chip (even with ethanol) increases proportionally with the chip size and pre-storage duration. This is because of gradual hydrophobic recovery (Hydrophilic loss) after PDMS and Glass plasma binding.

In the spinning chip, centrifugal buoyancy force and inertial forces (from alternating the chip spinning speed) are employed to remove the air bubbles from the surfaces (cell trap zone) and drive them towards the chip center. Alternating the chip spin speed between a low (15-25 Hz) and a high (30-50 Hz) rotational frequency creates adequate buoyant force to push the air bubble towards the chip center, and creates inertial forces to facilitate air bubbles drifting between the traps. The duration required for air bubble removal varies (20-60 sec) depending on the spin alternation speeds and acceleration used.

Contactless Liquid Delivery and Extraction on Cell Pairing and Fusion Chip.

Employing centrifugal force and central chamber enable contactless liquid pipetting and collection manually or automated by a robotic pipette as shown in the schematic in FIG. 11. A vacuum/suction nozzle at the top of the PDMS layer continuously collects waste solutions from the outlet ring.

High-Efficiency Cell Capturing Via Cell Sedimentation at Terminal Velocity

On the spinning chip, cells in the absence of liquid flow travel at a constant speed known as terminal velocity. The sedimentation of the cells directly towards cell trap opening is expected to increase cell capturing efficiency when compared with the flow-based method in which cells are propelled both towards and around the traps.

Using Both Centrifugal and Shear Forces to Deform and Trap Cells

In the chip, both centrifugal force from spinning and shear forces from liquid flow are employed to deform the cells into double-cell traps. When liquid is injected to the chamber and the chip is spun at high speeds (15 Hz>), the centrifugal force drags the liquid toward the outer periphery and the induced liquid flow and shear force is used for cell deformation/trapping step. The spinning speed defines the amount of liquid flow and centrifugal forces acting on the cells.

In a hybrid chip (FIG. 12), the rate of input liquid flow (and the resulting shear stress) can be controlled via the external pump that is connected to a stationary port of the rotary union. In other words, in the hybrid chip (using rotary union) the liquid flow rate can be adjusted independent of the chip rotational frequency.

Using Buoyant Force for Release and Collection of Cells

When a liquid with a high density is being introduced to the chip, elevating the rotational frequency increases the buoyant force applied on the cells, which is against the direction of the centrifugal force. Creating adequate cell buoyant force allows trapped cells (fused cells) to deform or squeeze-out of the traps and travel to accommodate in the central chamber for collection.

Sedimentation of Uncaptured Cells at Outer Rim of the Chip and their Manual or Automated Collection

In the spinning chip, cells that are not captured by cell traps are sedimented at the outer rim of the chip and can be collected right after the cell capture step. While the cell sedimentation at the outer rim occurs, the waste solution continuously flows to the upper chip layer towards opening of the plastic cover and can be collected by contactless vacuum nozzle. In the hybrid chip, a channel at the outer edge of the chip can be used to collect the sedimented cells.

Those skilled in the art will recognize, or be able to ascertain using no more than routine experimentation, many equivalents to the specific embodiments of the method and compositions described herein. Such equivalents are intended to be encompassed by the following claims.

We claim:

1. A microfluidic system for cell pairing and cell fusion, comprising
i) a transparent layer in a disc shape; and
ii) a support layer,
wherein the transparent layer comprises a plurality of cell traps and at least one inlet port for receiving a sample of liquid and cells in the center of the cell traps;
wherein the cell traps are arranged in one or more concentric circular arrays around the central inlet port forming a ring of a circular cell trap zone.

2. The microfluidic system of claim 1, wherein the area between the inlet port and the first circular array of cell traps comprises a plurality of support pillars, preferably arranged in one or more concentric circular arrays around the central inlet port.

3. The microfluidic system of claim 1, wherein the area between the inlet port and the first circular array of cell traps comprises a plurality of cell filters, preferably arranged in one or more concentric circular arrays around the central inlet port.

4. The microfluidic system of claim 1, wherein the cell trap comprises
i) a single-cell capture cup with a constriction,
ii) a double-cell capture cup, and
iii) one or more support pillars in contact with the support layer.

5. The microfluidic system of claim 4, wherein the opening of the constriction is similar or smaller than the diameter of a single cell to ensure capture of only one cell.

6. The microfluidic system of claim 4, wherein the support pillars are placed on both sides of the trap to increase the fractional flow rate through the traps.

7. The microfluidic system of claim 1, wherein the transparent layer has a diameter between about 2 cm and about 20 cm, inclusive, preferably between about 3 cm and about 10 cm, inclusive.

8. The microfluidic system of claim 1, wherein the ring of the circular cell trap zone has an inner diameter between about 1 cm and about 4 cm, inclusive, and an outer diameter between about 2 cm and about 10 cm, inclusive.

9. The microfluidic system of claim 1, wherein the microfluidic system has a total number of cell traps between about 100,000 and 1,000,000, inclusive, preferably between about 250,000 and 500,000.

10. The microfluidic system of claim 1, wherein the transparent layer and the cell traps are made from polydimethylsiloxane.

11. The microfluidic system of claim 1, wherein the support layer is made from glass or plastic, optionally with electrodes.

12. The microfluidic system of claim 1, wherein the microfluidic system further comprises a plastic cover attached to the support layer to create a reservoir for collection of cells and reagents passing through the chip.

13. The microfluidic system of claim 1, wherein the inlet port is connected to a robotic pipetting system for receiving a sample of liquid and cells.

14. The microfluidic system of claim 1, wherein the inlet port is connected to a rotary union for receiving a sample of liquid and cells and removing cells and reagents passing through the chip.

15. A method of pairing and fusing cells comprises the step of loading a first cell population onto the microfluidic system of claim 1, followed by loading a second cell population onto the microfluidic system, and then fusing cells of the first population with cells of the second population.

16. The method of claim 15, wherein the method comprises the step of priming the microfluidic system prior to loading of the first cell population.

17. The method of claim 16, wherein the priming step includes washing and filling the microfluidic system with cell culture media with minimal air bubbles in the microfluidic system.

18. The method of claim 15, wherein loading the first cell population results in a single cell of the first cell population inside the double-cell capture cup, and the loading of the second cell population results in a single cell of the second cell population inside the same double-cell capture cup.

19. The method of claim 15, wherein the cell fusion is electrically or chemically induced.

20. The method of claim 19, wherein the cell fusion is polyethylene glycol (PEG)-mediated cell fusion.

21. The method of claim 15, further comprising the step of collecting the fused cells from the microfluid chip.

22. The method of claim 15, wherein the cell fusion is between a dendritic cell and a tumor cell.

23. The method of claim 22, wherein the tumor cell is derived from renal cell carcinoma, prostate, or breast carcinoma.

24. The method of claim 22, wherein the dendritic cell is autologous.

25. The methods of claim 22, wherein the tumor cell is an allogeneic tumor cell.

26. The method of claim 22, further comprising the step of administering the fused cell of the dendritic cell and the tumor cell to a subject in need thereof.

27. The method of claim 15, wherein the cell fusion is between a B cell and a myeloma cell.
